(19) United States
(12) Patent Application Publication
(10) Pub. No.: US 2024/0024619 A1
(43) Pub. Date: Jan. 25, 2024

(54) METHOD AND SYSTEM FOR TREATING SLEEPING OR MOVEMENT DISORDER

(71) Applicant: BIOPRO SCIENTIFIC CO., LTD., HSINCHU CITY (TW)

(72) Inventors: YEN-CHUNG CHANG, HSINCHU CITY (TW); HSIN CHEN, HSINCHU CITY (TW); SHIH-RUNG YEH, HSINCHU COUNTY (TW); CHUNG-YAO HSU, KAOHSIUNG (TW)

(21) Appl. No.: 18/356,610

(22) Filed: Jul. 21, 2023

Related U.S. Application Data
(60) Provisional application No. 63/369,069, filed on Jul. 22, 2022.

(51) Int. Cl. A61M 21/02; A61B 5/372; A61B 5/389

(52) U.S. Cl. CPC A61M 21/02; A61B 5/372; A61B 5/389; A61M 2205/3303

(57) ABSTRACT

A method for treating sleeping or movement disorder is provided. The method includes the operations as follows. A brainwave of a patient with sleeping or movement disorder is recorded. An onset of a STW oscillation episode from the brainwave is identified, wherein the STW oscillation episode is a STW signal having an oscillation frequency in a range of from about 2 Hz to about 4 Hz. a first stimulation is delivered to the patient when the onset of the STW oscillation episode is identified from the brainwave. The first stimulation is adapted according to a measurable feature of the STW oscillation episode. A system for treating sleeping or movement disorder is also provided.

### PRIORITY CLAIM AND CROSS-REFERENCE

This application claims the benefit of prior-filed U.S. provisional application No. 63/369,069, filed on Jul. 22, 2022, and incorporates by reference herein in its entirety.

### FIELD

The present disclosure relates to a method and a system for treating sleeping or movement disorder, particularly, to a method and a device that employ sawtooth waves (STWs) oscillations episodes as a biomarker for predicting the risk of developing Parkinson's disease (PD) in patients with REM sleep behavior disorder (RBD).

### BACKGROUND

Dementia has been the most prevalent neurodegenerative disease, and has affected more than 50 million people worldwide. The Parkinson's disease (PD) is the second most prevalent neurodegenerative disease, impacting more than 8 million people globally. In the US, 60,000 cases are diagnosed annually, not including several thousand undiagnosed cases. While in the world, there are over 210,000 cases diagnosed each year. Both dementia and Parkinson's disease have significantly degraded the quality of life for a substantial population in this century. In addition, disorders such as depression, addiction, epilepsy, and motor neuron disease affect the youth and children in society, resulting in significant healthcare costs in every country. For instance, the US government has spent over 9.56 billion USD on treating Parkinson's disease and Alzheimer's disease. Therefore, the development of a medical device for early-stage diagnosis and intervention in these disorders is crucial.

### SUMMARY

It is one aspect of the present disclosure to provide a method for treating sleeping or movement disorder. The method includes the operations as follows. A brainwave of a patient with sleeping or movement disorder is recorded. An onset of a STW oscillation episode from the brainwave is identified, wherein the STW oscillation episode is a STW signal having an oscillation frequency in a range of from about 2 Hz to about 4 Hz. a first stimulation is delivered to the patient when the onset of the STW oscillation episode is identified from the brainwave. The first stimulation is adapted according to a measurable feature of the STW oscillation episode.

It is another aspect of the present disclosure to provide a system for treating sleeping or movement disorder. The system includes an electrode module, a neuromodulation module and a dock module wirelessly connected to the wearable neuromodulation module. The electrode module includes a plurality of electrodes, configured to obtain pathological activities of a patient with sleeping or movement disorder; and a neuromodulation module coupled with the plurality of electrodes, configured to record pathological activities and execute a stimulation through the plurality of electrodes simultaneously. The dock module is configured to optimize a parameter of the stimulation. The pathological activities comprises a sawtooth wave (STW) demonstrating at least one oscillation episode each having an oscillation frequency in a range of from about 2 Hz to about 4 Hz.

### BRIEF DESCRIPTION OF THE DRAWINGS

Aspects of the present disclosure are best understood from the following detailed description when read with the accompanying figures. It is noted that, in accordance with the standard practice in the industry, various structures are not drawn to scale. In fact, the dimensions of the various structures may be arbitrarily increased or reduced for clarity of discussion.

FIG. 1 illustrates a schematic diagram of a system for treating sleeping or movement disorder and the peripheral devices according to some embodiments of the present disclosure.

FIG. 2 illustrates flow chart of a method for treating sleeping or movement disorder according to some embodiments of the present disclosure.

FIG. 3A illustrates a block diagram of a system for treating sleeping or movement disorder according to some embodiments of the present disclosure.

FIG. 3B illustrates a block diagram of a system for treating sleeping or movement disorder and the path of data obtained by the electrodes according to some embodiments of the present disclosure.

FIG. 3C illustrates a block diagram of a device for treating sleeping or movement disorder and the information paths including the transmission of optimized parameter according to some embodiments of the present disclosure.

FIG. 3D illustrates a circuit diagram of a system for treating sleeping or movement disorder according to some embodiments of the present disclosure.

FIG. 3E illustrates a block diagram of a central control submodule according to some embodiments of the present disclosure.

FIG. 4 illustrates a block diagram of a plurality of PCBs used in a device for treating sleeping or movement disorder according to some embodiments of the present disclosure.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawings.

### DETAILED DESCRIPTION

The following disclosure provides many different embodiments, or examples, for implementing different features of the provided subject matter. Specific examples of elements and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Further, spatially relative terms, such as “beneath,” “below,” “lower,” “above,” “upper”, “on” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

As used herein, the terms such as “first”, “second” and “third” describe various elements, components, regions, layers and/or sections, these elements, components, regions, layers and/or sections should not be limited by these terms. These terms may be only used to distinguish one element, component, region, layer, or section from another. The terms such as “first”, “second”, and “third” when used herein do not imply a sequence or order unless clearly indicated by the context.

Parkinson's disease (PD) is primarily caused by the degeneration of dopaminergic neurons that project from the substantia nigra pars compacta (SNpc) to the striatum. By the time PD patients start displaying obvious motor symptoms, over 50% of the SNpc neurons have already degenerated. Consequently, identifying Parkinsonian patients in the early stages becomes challenging, and they often miss opportunities for early diagnosis and treatment.

Two methods have been developed to detect the degeneration of SNpc neurons during the early onset of PD. One method involves imaging the density of dopamine transporters (DAT) in the striatum using 99 mTC-TRODAT SPECT (single-photon emission computerized tomography). Although this examination has high accuracy, its cost is prohibitively expensive for most individuals without Parkinsonian symptoms to consider self-funding the procedure. The other method involves examining the concentration of D-synuclein protein in the blood-cerebrospinal fluid. While this method is simpler and less costly, the specificity and sensitivity of the blood test remain debatable. Consequently, clinical practice continues to rely on physical examination and questionnaires to diagnose or assess the progression of PD and evaluate the efficacy of medication. It is important to note that medication does not halt the progression of PD and often induces numerous side effects.

Deep brain stimulation (DBS) has been identified as a promising alternative to medication for treating PD and other neural disorders. By precisely applying electrical stimulation to brain regions such as the subthalamic nucleus (STN) through implanted electrodes, DBS effectively regulates abnormal nerve activity and improves movement impairment in PD patients. Since the FDA approved DBS for treating advanced PD in 1997, it has become the primary alternative for PD treatment, particularly in addressing side effects and drug resistance issues that arise from long-term medication. For instance, DBS therapy is covered by health insurance if a PD patient has been diagnosed for more than five years and is unable to continue medication due to adverse reactions from prolonged use.

In 2016, the FDA further approved DBS for treating early-stage PD. However, due to the high surgical risk of implanting deep-brain electrodes and other side effects resulting from continuous DBS, most PD patients prefer medication as the main treatment during their early stages. In other words, the application of DBS still faces two major challenges: (1) invasive treatment involving high-risk surgery; and (2) limited timing of application primarily intended for advanced PD cases.

The rTMS is expected to get FDA approval for treating the PD in the future. However, the TMS requires high electrical currents to generate large enough magnetic stimulation. This requirement unavoidably leads to a bulky size for the TMS coil, and the TMS can only be operated by trained healthcare professionals. Contrary to the TMS, the tES generate bipolar stimulation with a direct current or an alternating current through the electrodes placed on the head. The current flowing trans-cranially through the brain subsequently modulates neuron activities in the brain. As the tES does not require a large current to generate magnetic stimulation, the size of the tES device is much more compact and portable. The FDA has approved the application of the tES to treating depression, migraine, autism, etc.

In recent years, non-invasive neuromodulation has gained increasing attention in clinical studies alongside invasive DBS. For instance, transcranial magnetic stimulation (TMS) and transcranial electrical stimulation (tES) can be utilized in numerous clinical trials. Furthermore, applying repetitive TMS (rTMS) to the motor or frontal cortex has shown significant improvement in Parkinsonian motor symptoms and the suppression of dyskinesia induced by Levodopa-like medication. It is anticipated that rTMS will receive FDA approval for treating PD in the future. However, TMS requires high electrical currents to generate sufficient magnetic stimulation, resulting in a bulky coil size, and such technique can only be operated by trained healthcare professionals.

On the other hand, tES generates bipolar stimulation using direct or alternating current through electrodes placed on the head. The current flowing through the brain transcranially subsequently modulates neuron activity. Since tES does not require high current for magnetic stimulation, the tES device is much more compact and portable. In fact, tES can be used for treating depression, migraines, autism, and other conditions.

Generally, tES can be further categorized into at least two types: transcranial direct current stimulation (tDCS) and transcranial alternating current stimulation (tACS). Applying tDCS to the motor cortex or prefrontal cortex can achieve similar therapeutic effects to TMS, while such technique also helps improve unbalanced gait and bradykinesia in PD patients. Accordingly, it is believed that tES is potentially as an alternative to TMS. Moreover, due to the bidirectional connections between the cerebellum and basal ganglia through the thalamus and pontine nucleus, as well as the favorable location of the cerebellum for tES application, it is possible to apply tDCS to the cerebellar region for treating PD, myasthenia gravis, tremors, and other conditions. However, inconsistent conclusions have been drawn from different clinical trials regarding the efficacy of tDCS in improving dyskinesia and dystonia in PD. This inconsistency mainly stems from the fact that optimal stimulation sites and waveforms differ among patients. In addition, electroencephalography (EEG) recording before and after tES application can guide the determination of optimal location and adaptation of tES. Assuming tES can effectively treat PD, it is crucial to identify an economical and reliable method for diagnosing early-stage PD patients.

Regarding REM sleep behavior disorder (RBD), it is observed that 50-80% of RBD patient's progress into PD or other neurodegenerative disorders, such as multiple system atrophy and Lewy-body dementia, within 5-10 years. These findings enable the diagnosis of PD before the onset of any motor symptoms. The primary neurophysiological manifestation of RBD is the macrostructure abnormality in REM sleep, also known as electromyography (EMG) without muscle atonia (RWA), which serves as the standard for diagnosing RBD using polysomnography (PSG). However, the clinical and academic communities now agree that RBD should no longer be referred to as the pre-stage of Parkinson's disease, but rather as the non-motor stage of PD. Unfortunately, neither physicians nor neuroscientists have identified a reliable method to determine which subgroup of RBD patients will develop PD. In other words, there is no established biomarker for predicting the risk of PD development in patients with RBD. Consequently, the opportunity to slow down the progression of PD in its early stages remains significantly limited.

EMG and electro-oculography (EOG) are the two electrophysiological signals with the highest amplitude and the lowest sensitivity, and they can easily interfere with other electrophysiological signals. On the other hand, EEG is the electrophysiological signal with the lowest amplitude and highest sensitivity, but it is also easily susceptible to interference. The changes in the sleeping microstructure represented by the EEG of RBD patients, such as power spectral density, cyclic alternating pattern (CAP), and sleep-spindle density, can begin before any abnormalities are observed in the EMG. These changes are difficult to discern through visual observation during sleep-stage interpretation in a sleep monitoring center.

According to the standards published by the American Academy of Sleep Medicine, human sleep can be divided into five different stages: awake, Phase I, Phase II, slow-wave, and rapid eye movement (REM). In a typical sleep cycle, Stage I accounts for about 5%, Stage II for about 45%-55%, slow-wave sleep for 20%-26%, and REM for about 20%-25%. If the non-REM period and the subsequent REM period are considered as one sleep cycle, a full night of sleep usually consists of 4 to 6 cycles, with each cycle lasting about 90 minutes.

In addition to the duration of different sleep stages, the proportions of different stages and the structure of a sleep cycle are often altered by various sleep disorders or other neural and mental illnesses. The REM and slow-wave periods are particularly sensitive to the development of these diseases. For example, patients with depression often experience shortened REM latency (the time between falling asleep and the onset of the first REM sleep period) and increased REM density. On the other hand, PD is associated with an increase in REM latency and a decrease in REM density. It is known that PD may disrupt the continuity of sleep and alters sleep structure, and the power of theta waves may significantly reduce in PD patients. Therefore, it is possible to diagnose the non-motor stage of PD by monitoring the sleeping EEGs of patients and develop algorithms for automatically detecting alterations in sleep structure, as well as biomarkers related to early-stage PD.

A healthy human EEG may exhibit specific patterns of activity that are correlated with the individual's level of wakefulness. These patterns fall within the frequency range of 1 to 30 Hz, with amplitudes varying between 20 and 100 μV. The frequencies of these patterns/waves can be categorized into different groups: the alpha-band (8-13 Hz), the beta-band (13-30 Hz), the delta-band (0.5-4 Hz), and the theta-band (4-7 Hz). It is important to note that the frequency boundaries of these common rhythmic neuronal activities are not universally agreed upon in academia and may vary slightly in different literature sources. Generally, alpha waves are observed during relaxed wakefulness and are most prominent over the parietal and occipital sites. On the other hand, beta waves are more prominent in frontal areas and other regions during intense mental activity. When a relaxed individual is instructed to open their eyes, alpha activity decreases while beta activity increases.

Regarding the oscillations observed by EEG, the beta-band (13-30 Hz) oscillation (or called beta oscillation) in the cortico-basal ganglia circuit has been observed in both PD patients and Parkinsonian rat models. The oscillation, such as the beta-band oscillation, is mainly attributed to synchronized neural activity in the cortico-basal ganglia circuit. Adaptive DBS, which aims to suppress the beta-band oscillation, has been experimented with in both human and animal models. Experimental data indicate that adaptive DBS is more effective than open-loop DBS in ameliorating motor deficits. However, there are still several debates surrounding beta-band oscillation. For example, firstly, the beta-band oscillation consistently occurs in the cortico-basal ganglia circuit for PD subjects and resumes as soon as the stimulation is turned off. Therefore, continuous stimulation seems essential for suppressing the beta-band oscillations indefinitely. In other words, if the pathological activity to be suppressed is beta oscillation, closed-loop control should focus on identifying an optimal stimulation level to lower the power of the beta-band oscillation to a target level, rather than triggering the stimulation at precise timing. Secondly, the energy level of the beta-band oscillation is not found to be fully correlated with the extent of motor deficits in several experiments. Thirdly, the beta-band oscillation emerges after the significant loss (>30%) of dopaminergic neurons. That is, suppressing the beta-band oscillation is useful only for advanced PD patients and does not help slow down the degeneration of dopaminergic neurons.

Neurons in the STN have been found to exhibit burst firing with significantly longer bursting duration, which correlates with motor symptoms such as bradykinesia or rigidity. Pathological bursting activity can be induced by hyperpolarizing STN neurons and has been shown to cause normal rats to exhibit hypokinetic behaviors similar to Parkinsonian rats. These findings suggest that suppressing bursting activity in STN neurons can help alleviate movement deficits. However, this pathological signature relies on single-unit recording, which is challenging to achieve chronically in the STN due to its small size and deep location within the brain. Furthermore, it does not guarantee that recorded neurons will exhibit bursting behavior. Additionally, detecting and analyzing bursting behavior requires a higher sampling rate and more sophisticated algorithms for spike detection and pattern recognition.

The third type of pathological activity observed in Parkinsonian animal models is high-voltage spindles (HVS). HVS is a spike-and-wave oscillation that propagates in the cortico-basal ganglia circuit, particularly in the early stages of Parkinson's disease. HVS is result from the depletion of dopaminergic neurons projecting from SNpr to the striatum. HVS is a non-stationary, stochastic neural oscillation that typically lasts for 2-4 seconds in each episode. It can also be suppressed by applying deep brain stimulation (DBS) to the STN. Due to the random occurrence of HVS, precise timing control of DBS is crucial to maximize stimulation efficacy while minimizing side effects.

HVS has received less attention than beta-band oscillation because HVS has not been widely reported to occur in PD patients, and HVS occurs with a significant higher likelihood in the early stages of PD, making invasive, chronic recording in patients during that stage nearly impossible. Additionally, HVS is a nonstationary signal that can be easily missed, and its occurrence becomes less frequent as PD progresses.

In some embodiments of the present disclosure, the feature of sawtooth waves (STWs) is utilized as a biomarker for predicting the risk of developing PD in patients with RBD. Specifically, the HVS mentioned earlier predominantly occurs during REM sleep in rat models, which bears resemblance to STWs observed in human REM sleep. Therefore, the present disclosure employs the STWs feature as a biomarker to monitor the dysregulation of human REM sleep. By assessing the decrease in STWs, alterations in the proportion and power of EEG fast and slow activities, and augmented EMG activity (RSWA or REM sleep without muscle atonia), the sleeping or movement disorders such as PD, alpha-synucleinopathies (ASP), or RBD can be predicted and treated accordingly.

Furthermore, STW is kind of sporadic oscillation episode having spike-and-wave patterns in EEG. STW has a frequency within the range of 2-4 Hz, which is overlapped with the delta-band oscillation (i.e. 0.5-4 Hz), however, STW is different from delta-band oscillation since STW in the aspects of duration and randomness. That is, STW occurs as an “oscillation episode”, which is a series of sudden oscillations having beginning and end of each episodes, whereas the delta-band oscillation is a continuous signal pattern without beginning and end. Generally, each STW oscillation episode has an oscillation duration from about 1 to about 10 seconds.

As aforementioned, STWs can be observed in human REM sleep, similar to how high-voltage spindles (HVS) occur during REM in animals. However, in patients with sleep disorders or Parkinson's disease, STW may be abnormally observed during the other sleeping stages (i.e., the stages other than the REM stage during sleeping) or even during wakefulness (i.e., an awake stage). This indicates a disruption in physiological rhythms of these patients. Therefore, when applying closed-loop stimulation, it is important to determine whether STW occurs during REM stage in order to decide whether stimulation should be applied to suppress it. In some scenarios, although STW occurs normally during REM, the frequency of occurrence is abnormally high. In such scenarios, a (lower) threshold value can be established based on the average occurrence of STW in REM among normal individuals as a reference. Once the frequency exceeds this threshold value (i.e., a predetermined occurrence frequency), stimulation can be applied to suppress the excessive and abnormally frequent STW. In some scenarios, when determining STW occurs during other sleeping stages as previously provided or during wakefulness (i.e., awake stage), stimulation should be delivered at the onset of such STW in order to suppress the STW occurs under such conditions. In other words, in some embodiments of this disclosure, the goal of the stimulation includes not only to conditionally suppress the STW which occurs during the REM stage when it exceeds a predetermined occurrence frequency, but also to alter the timing of STW occurrence, e.g., completely suppressing the STW during other sleeping stages and during awake stage, so that the STW may only occur during the REM stage.

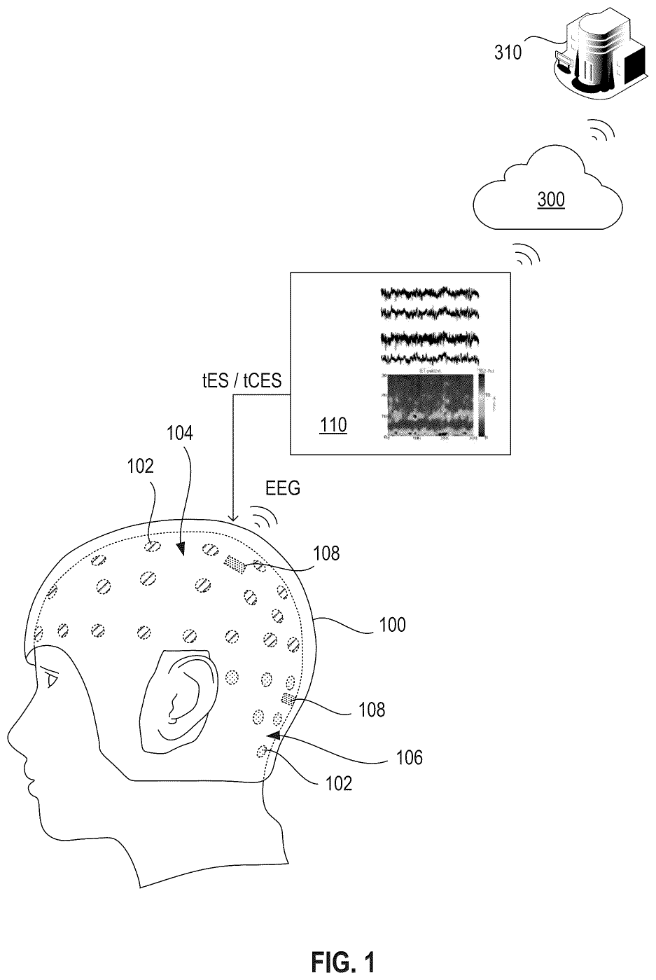

In some embodiments, as shown in FIG. 1, a wearable (medical) device 100 is utilized for diagnosing and treating early-stage sleeping or movement disorders such as PD using transcranial electrical stimulation (tES) or transcutaneous electrical stimulation (tCES), with stimulation adapted according to EEG recordings. In some embodiments, the wearable device 100 takes the form of a hat which includes a plurality of electrodes 102 placed on both the cerebral region 104 and the cerebellar region 106 of the brain. In some embodiments, the wearable device 100 can record EEG and deliver tES or tCES through each electrode 102. Other than the electrodes 102, the wearable medical device 100 may have one or more neuromodulation module 108 configured to process signals and communicate with other components, such as a neuromodulation controller 110. In some embodiments, the neuromodulation controller 110 can receive the patient's EEG from the wearable device 100 and use it to detect/identify biomarkers (e.g., STW oscillation episodes) and to monitor the patient's sleep structure. This allows the adapted tES and/or tCES to be delivered to the patient when necessary. Additionally, in some embodiments, the neuromodulation controller 110 can be connected to a secure cloud data service 300. This enables the sharing of the treatment process and the patient's status with hospitals or clinics, allowing these professional medical departments 310 to provide timely assistance when necessary. More details of the neuromodulation module 108 and the neuromodulation controller 110 will be described later.

In some embodiments, the stimulation (e.g., tES or tCES) can be delivered through one or more selected pairs of electrodes 102 in bipolar mode, where one subset of electrodes functions as the anode and the other subset functions as the cathode.

The technique of tES includes transcranial direct current stimulation (tDCS) and transcranial alternating current stimulation (tACS). The tDCS is a form of neuromodulation that uses a constant, low direct current delivered via electrodes on the head, while the tACS is another non-invasive stimulation method that uses alternating current. In some embodiments, tES can be performed by using two different frequencies (e.g., E₁(f) and E₂(f+Δf)) of sine waves to achieve deeper brain region stimulation through temporal interference, while avoiding stimulation interference in shallow brain regions. In such embodiments, constructive interference of the selected sine waves with different frequencies may happen at the deeper brain region to stimulate such region specifically. In other embodiments, the phase synchronized tACS can be applied.

In some embodiments, voltage stimulation is applied during the stimulation process, involving the application of a voltage difference across the anode and cathode electrodes to induce current flow between the electrode sets. In other embodiments, current stimulation is applied during the stimulation process, wherein a predefined current is delivered to flow between the electrode sets.

In some embodiments, the wearable device 100 allows users to randomly select one or more pairs of electrodes 102 (i.e., the anode-cathode pairs), with each pair having an independent stimulation waveform. In some embodiments, the wearable device 100 records EEG before, during, and after stimulation, enabling medical professionals to adjust the stimulation pattern for each patient. Additionally, the wearable device 100 can detect PD-related biomarkers and automatically adapt the stimulation pattern accordingly.

As aforementioned, the STW is an oscillation episode that randomly occurs in the EEG recording and can be utilized as a biomarker in treating PD, particularly, in the early stages thereof. The frequency of the STW signal ranges from approximately about 2 to about 4 Hz, and each STW oscillation episode has an oscillation duration from about 1 to about 10 seconds.

In other words, from a technical point of view, the STW oscillation episodes observed in the scalp EEG of patients with RBD are considered as a biomarker for early-stage sleeping or movement disorders. In this disclosure, these STW signals can be compared to the pathological HVS signals observed in Parkinsonian rat models. Since HVS signals can be suppressed by DBS, the wearable device 100 shown in FIG. 1 will automatically record the scalp EEG to monitor the occurrence of STW oscillation episodes using machine-learning algorithms. Once a STW signal is detected, the wearable device 100 will generates tES or tCES to modulate the occurrence of STW signals in patients, aiming to suppress or modulate the occurrence timing of STW. As aforementioned, the ultimate goal is to alter the timing of STW occurrence so that it only happens in normal frequency during REM, rather than completely suppressing all STW. By doing so, the neuromodulation in the present disclosure may thus slow down or intervene in the progression of sleeping or movement disorders, including RBD, ASP, or PD, especially in their early stages.

In some embodiments, the stimulation is automatically triggered at the onset of the STW oscillation episodes. The stimulation pattern and location are then adapted for each individual STW oscillation episode based on their effectiveness in modulating the occurrence of STW oscillation episodes or modifying sleeping rhythms.

In some embodiments, the adaptable stimulation parameters include the stimulation waveform, the stimulation frequency, the stimulation amplitude, the stimulation duration or repetitiveness, the location of the stimulation electrodes (sites), and any of them may be changed according to the measurable feature of the oscillation episode.

In some embodiments, the measurable feature of the oscillation episode may be recorded before or during delivering the first stimulation, and the measurable feature obtained may thus be used for adapting the first stimulation. In some embodiments, the measurable feature includes an oscillation intensity and an oscillation frequency. In some embodiments, the measurable feature may further include an oscillation duration of the STW oscillation episode and a frequency of occurrence of the STW oscillation episode within a predetermined timeframe.

To be more detailed, there are different approaches to achieve the adaptation of the first stimulation. This means that more than one stimulation form can be determined based on the measurable feature. In some embodiments, the stimulation form can include a stimulation frequency, a stimulation intensity, and a stimulation waveform, and any of these can be changed based on the measurable feature of the oscillation episode. The stimulation intensity may include the amplitude of the stimulation. Additionally, the stimulation form may also include a stimulation duration and a frequency of occurrence of the stimulation.

In some embodiments, the measurable feature can be the energy derived from a single STW oscillation episode. For example, the first stimulation can be adapted from a higher stimulation intensity to a lower stimulation intensity once the energy derived from the STW oscillation episode is measured to be half of its original value (e.g., at the onset of the STW oscillation episode). Another example is changing the stimulation intensity once the oscillation intensity is measured to be one-third of its original value (e.g., at the onset of the oscillation episode).

The measurable features for adapting the stimulation can be acquired through different approaches depending on their nature. For example, the oscillation intensity and frequency can be recorded in real-time for determining real-time feedback and adapting the stimulation accordingly. On the other hand, the oscillation duration of the STW oscillation episode and the frequency of occurrence of the STW oscillation episode can be determined within a predetermined timeframe, such as a predetermined time window when multiple STW oscillation episodes occur. This information can be used to determine an average feedback for the predetermined timeframe. In some embodiments, the average feedback may not lead to real-time adaptation of the stimulation but may be used to determine the subsequent stimulation form. The oscillation duration of the STW oscillation episode can be determined by averaging the durations of at least three STW oscillation episodes, two STW oscillation episodes, or a single STW oscillation episode. The frequency of occurrence of the STW oscillation episode can be calculated within a suitable timeframe, such as one hour, 30 minutes, or 15 minutes.

When using the oscillation duration as a measurable feature for adapting stimulations, the first stimulation can be adapted from a longer stimulation duration to a shorter stimulation duration once the oscillation duration is measured to be shorter than a predetermined reference level, such as about 50% shorter than its original averaged value (e.g., the averaged oscillation durations within a specific timeframe). Generally, the oscillation duration may be significantly shortened after the stimulation, for example, from about 3.0 seconds (prior to applying stimulation) to less than about 1.0 second (after applying stimulation). In some embodiments, the stimulation may be applied until the STW signal is no longer observed on the recording, resulting in a comparatively long stimulation duration. In other embodiments, the stimulation can be adapted, paused, or terminated when the oscillation is still observable on the recording, and the STW signal may be suppressed instantly after the stimulation is adapted, paused, or terminated, thereby adopting a comparatively short stimulation duration. In some embodiments, the first stimulation duration is applied for a longer stimulation duration, and the follow-up stimulation durations may be adapted to be about 70% or even 80% shorter than the first stimulation duration. In some embodiments, the follow-up stimulations can be automated by the closed-loop system described herein with constantly-adapting stimulation duration based on various factors.

When using the frequency of occurrence of the STW oscillation episode as a measurable feature for adapting stimulations, the intensity of the first stimulation can be adapted to be greater once the frequency of occurrence of the STW oscillation episode is greater than a predetermined reference level, such as about 50% greater than its original averaged value (e.g., the averaged frequency of occurrence of the STW oscillation episodes within a specific timeframe).

In some embodiments, prior to applying the first stimulation to the patient, a reference level calibration or baseline calibration may be conducted when the measurable feature involves the STW oscillation duration and/or the frequency of occurrence of the STW oscillation episode of the brainwave.

In some embodiments, the stimulation waveform varies depending on the type of stimulation. For example, the stimulation waveform is constant for transcranial direct current stimulation (tDCS), is sinusoidal for transcranial alternating current stimulation (tACS), and is pulsed for transcutaneous electrical stimulation (tCES).

In some embodiments, the stimulation frequency can in the range of from about 100 Hz to about 180 Hz. In some embodiments, the stimulation frequency is in the range from about Hz to about 10K Hz. In some embodiments, the stimulation frequency depends on the demand of different types of treating purposes or clinical treating progresses and thus not limited to the ranges as aforementioned. In some practical examples, the stimulation frequency can be about 110 Hz.

In some embodiments, the stimulation amplitude can in the range from about 5 μA to about 4 mA with an adjustable step size of 5 μA/step for each channel.

In some embodiments, the duration of applied stimulation can be correlated with the duration of the observed STW oscillation episodes. For example, the stimulation can be terminated after the intensity of STW signal is decreased to a threshold value. In some embodiments, the stimulation can include multiple intermittent sub-stimulations during a single STW oscillation episode. For instance, the stimulation can be provided at a frequency of one second of stimulation followed by half a second of pause during the duration of each STW oscillation episode, reducing the overall dose of stimulation during each STW oscillation episode and thus minimizing potential side effects. In some embodiments, the pause during the duration of the STW oscillation episode can be used to evaluate whether the intensity of STW signal is decreased to a threshold value. For example, the stimulation can be paused after delivering for 0.2 second, and if the STW signal does not show an instantaneous value decayed to a predetermined lower threshold, the stimulation can be resumed, i.e., ending the pause state, for a duration of another 0.2 second, and so on, before the stimulation is terminated when the STW signal is decayed to the lower threshold, terminated, or after the STW signal being terminated for several seconds (e.g., 1.0 to 2.0 seconds, 1.0 to 3.0 seconds, or 2.0 to 3.0 seconds).

In some embodiments, the stimulation electrodes or the sites of the stimulation electrodes can be arranged as a pair or multiple pairs of electrodes 102 on the wearable device 100.

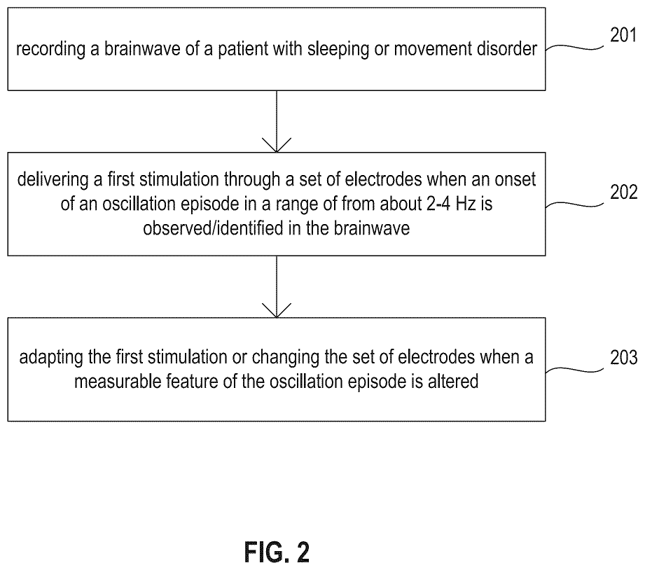

FIG. 2 illustrates the concept of closed-loop control on stimulation parameters. In some embodiments, the treatment based on the closed-loop control includes: (a) operation 201: recording a brainwave (e.g., central nervous signals) of a patient with sleeping or movement disorder (e.g., RBD, which has a high potential to progress into PD or other neurodegenerative disorders causing mobility impairment); (b) operation 202: delivering a first stimulation (e.g., tES or tCES) through a set of electrodes when an onset of an oscillation episode in a range of from about 2-4 Hz is observed in the brainwave; and (c) operation 203: adapting the first stimulation or changing the set of electrodes when a measurable feature of the oscillation episode is altered.

In some embodiments, the closed-loop treatment can be continuously carried out to delay the progression of sleeping or movement disorders alpha-synucleinopathies, such as PD, ASP, or RBD. In other embodiments, the duration of closed-loop treatment may be less than 2 hours per day to observe a sufficient effect. The latter option may offer an energy-efficient alternative for the patient. Considering that stimulation is a powerful force that strongly affects the cells and tissues near the stimulation area, controlling the daily stimulation period may help reduce the side effects of excessive brain stimulation.

To be more detailed regarding the closed-loop treatment illustrated in FIG. 2, in operation 201, at least the EEG signal can be recorded from patient's brainwave, and thus the features associated with pathological signatures (such as waveform or the intensity or occurrence timing of STW oscillation) can be extracted and detected in real-time. Once the pathological signature is detected, the first stimulation can be delivered in operation 202, and so that the brain may receive the first stimulation with pre-defined parameters. In some embodiments of the present disclosure, the brainwave activity is continuously recorded during and after stimulation, and features of pathological signatures are derived in real-time to adapt or adjust the stimulation parameters, as in the operation 203. Additionally, in some embodiments, other than EEG, both EMG and EOG can also be recorded simultaneously to confirm the necessity of triggering stimulation and optimize stimulation parameters.

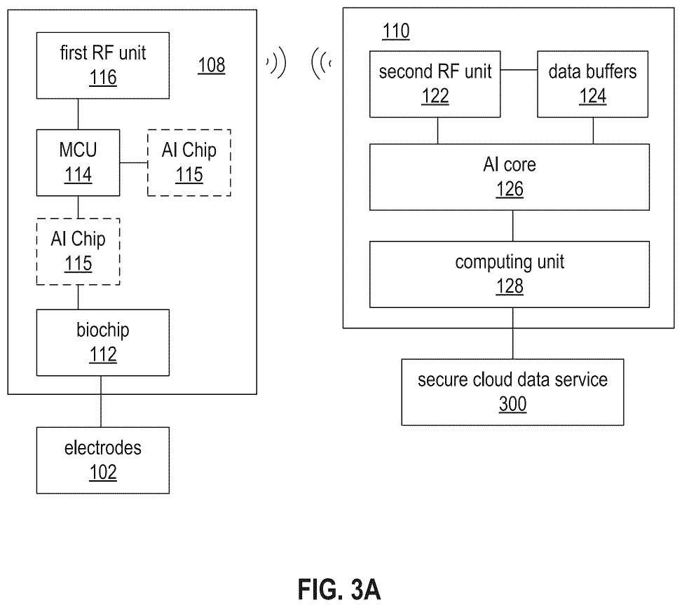

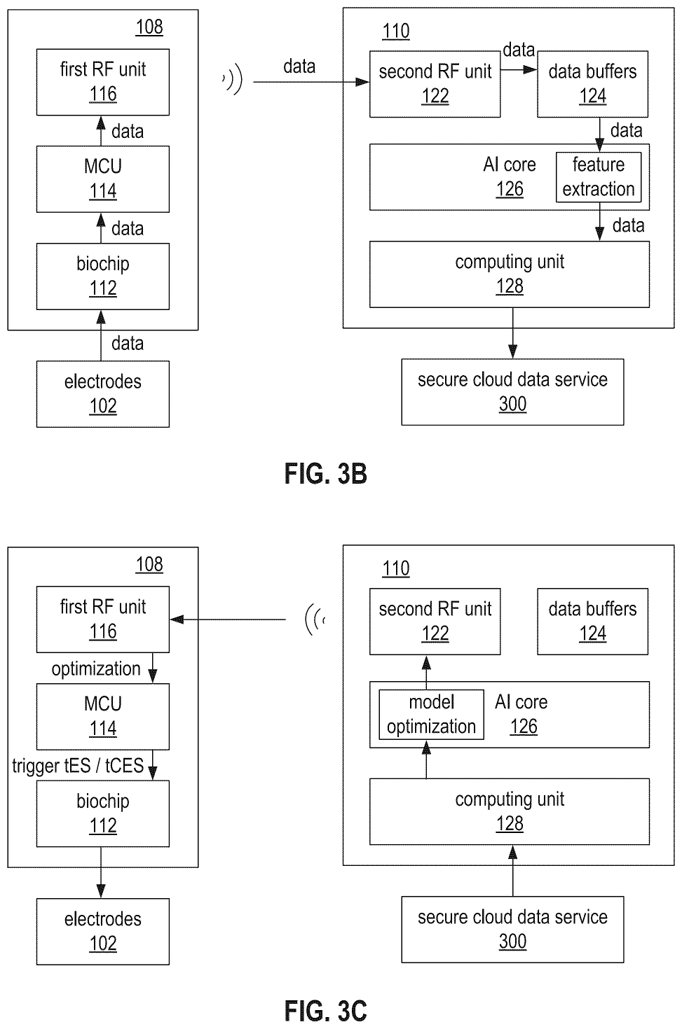

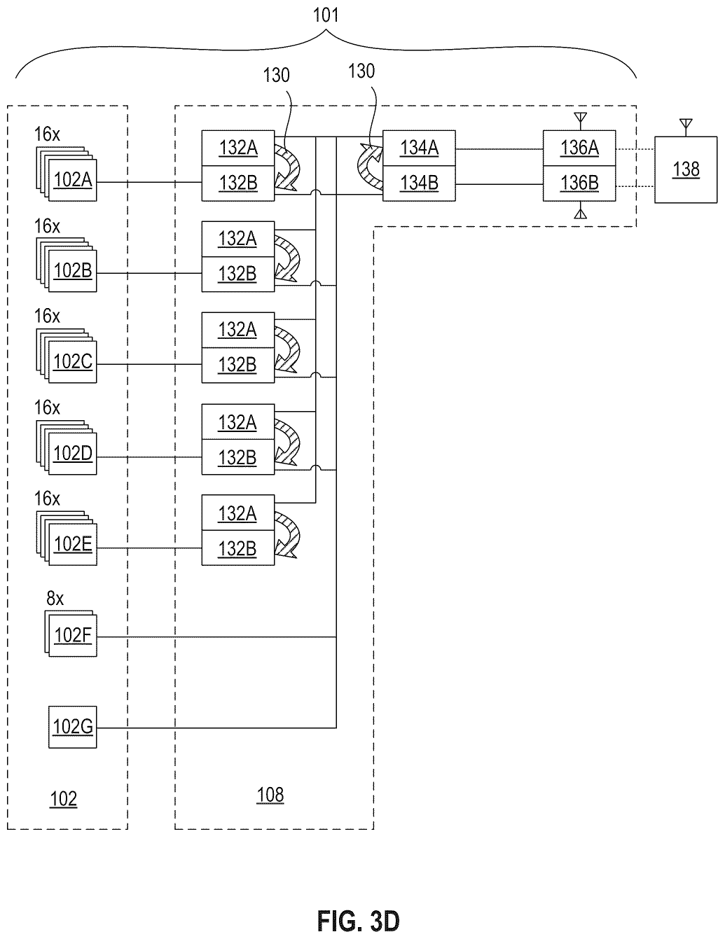

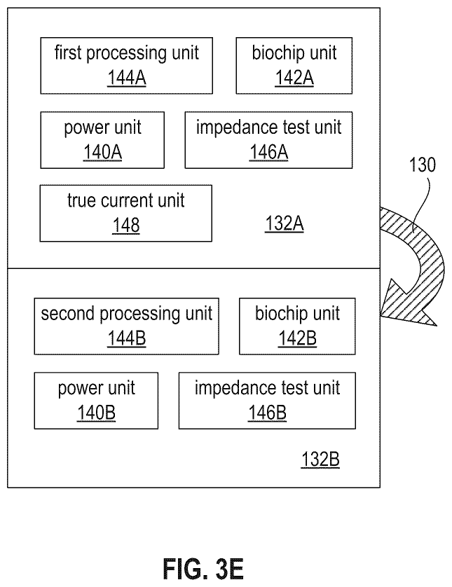

In some embodiment, the closed-loop treatment can be achieved through machine-learning algorithms. On other embodiments, the closed-loop treatment can be achieved through conventional signal processing algorithms (e.g., regression, time-frequency analysis, wavelet transform, etc.). FIG. 3A illustrates the hardware structure for implementing closed-loop stimulation according to some embodiments of the present disclosure.

In some embodiments, as shown in FIG. 3A, the hardware structure for implementing closed-loop stimulation includes the wearable device 100 previously illustrated in FIG. 1, and the neuromodulation controller 110 communicated with the wearable device 100.

In some embodiments, the neuromodulation module 108 of the wearable device 100 includes one or more biochips 112, a microcontroller unit (MCU) 114, and a first RF unit 116. The biochip 112 is coupled with the electrodes 102 that connected with the neuromodulation module 108. The MCU 114 is coupled within the biochip 112. The first RF unit 116 is coupled with the MCU 114 and is employed as an I/O configured to communicate with the neuromodulation controller 110 wirelessly through its transport (Tₓ)/Receive (Rₓ) functions.

In some embodiments, the neuromodulation controller 110 includes a second RF unit 122, a data buffer 124, an artificial intelligence (AI) core 126, and a computing unit 128. The neuromodulation controller 110 may be connected to a cloud server (e.g., the secure cloud data service 300 shown in FIG. 1) either through wired or wireless means. The second RF unit 122 is coupled with the data buffer 124. The AI core 126 is coupled with the second RF unit 122 and the data buffer 124. The computing unit 128 is connected to the AI core 126. As previously mentioned in describing FIG. 1, the neuromodulation controller 110 can connect to the secure cloud data service 300 and the connection enables the sharing of the treatment process and the patient's status with hospitals or clinics. These professional medical departments 310 can then provide timely assistance when necessary. For example, physiologists in hospitals or clinics can adjust the stimulation protocol and prescription. In some embodiments, advanced data services can be provided by the secure cloud data service 300.

The hardware structure illustrated in FIG. 3A may include the following key features: (a) a powerful MCU 114 is incorporated into the neuromodulation module 108; and (b) the AI core 126 based on the field programmable gate array (FPGA), graphical processing unit (GPU), or similar solutions is incorporated into the neuromodulation controller 110.

In some embodiments, the biochip 112 used in the wearable device 100 for the closed-loop treatment can record neural signals and generate stimulation through electrodes in the wearable device 100. In some embodiments, the MCU 114 in the wearable device 100 may read signals from the biochip 112 at a high sampling rate (e.g., 20k Sa./s per channel) and executes machine-learning algorithms (e.g., a first machine-learning process) to detect pathological activities in real-time. In some embodiments, the sampling rate per channel can be adjusted within a range of from about 100 to 20,000 Sa./s. Once the onset of a biomarker (i.e., the STW oscillation oscillation) is detected or identified, the MCU 114 can trigger the biochip 112 to generate tES or tCES, effectively suppressing pathological activities with negligible latency or modulating neural activities.

In some embodiments, the closed-loop treatment is executed directly in the wearable device 100, resulting in a delay between detection and stimulation expected to be smaller than 10 ms. As the neuromodulation controller 110 illustrated in FIG. 3A, the AI core 126 in the neuromodulation controller 110 is placed between the second RF unit 122 and the computing unit 128. In some embodiments, the AI core 126, such as the FPGA, may facilitate parallel, real-time processing of multi-channel data and execute advanced machine-learning algorithms (e.g., a second machine learning process) to regularly optimize the parameters of the algorithms in the wearable device 100 (e.g., the parameters of the stimulations). Since the AI core 126 is not disposed with the patient, there is less power consumption that needs to be considered. Therefore, the ability to perform machine-learning algorithms can be higher in the AI core 126 compared to the MCU 114 in the neuromodulation module 108.

For example, an adaptive autoregressive algorithm can be derived for detecting the onset of STW oscillation in real time, and an online learning algorithm based on the Kalman filter is applied for maintaining reliable detection from one day to another or for different subjects (i.e., among different durations or patients). In this example, the strategy is to implement the autoregressive algorithm in the MCU 114 in the neuromodulation module 108, while realizing the computationally-intensive Kalman filter in the AI core 126 of the neuromodulation controller 110.

In some embodiments, the MCU 114 can simply execute multiplication and accumulation of the signals from the biochip 112 to predict the onset of STW oscillation episode in real-time, while the AI core 126 learns optimal parameters and transmits new optimal values to the MCU 114 at regular intervals (e.g., every 10 minutes).

In some embodiments, the neuromodulation module 108 can further include an AI chip 115 connected with the MCU 114. Because MCU 114 may not have sufficient computational power to process the record data and stimulation data from and to the biochip 112 in real-time, in some embodiment, additional FPGA or AI chips 115 can be optionally utilized as an AI core and connected to the MCU 114 to enhance parallel computation power, as illustrated in the functional block of dotted lines. In some embodiments, the FPGA or AI chips 115 is arranged to receive or deliver data to the MCU 114. This arrangement can ensure the data transmitted therebetween possess a consistent format. In some embodiments, the FPGA or AI chips 115 is arranged to receive data from or deliver data to biochip 112 at one terminal and to deliver data to or receive data from MCU 114 at another terminal. This arrangement can provide instantaneous parallel computation power prior to delivering the data to MCU 114 and further lessen the computation burden of the MCU 114.

FIGS. 3B and 3C are used to illustrate the data communication within and between the neuromodulation module 108 and the neuromodulation controller 110.

In some alternative embodiments, as shown in FIG. 3D, the system for treating sleeping or movement disorder can include an electrode module 101 and a dock module 138. In some embodiments, the electrode module 101 is a wearable device that substantially includes the electrodes 102 and the neuromodulation module 108 previously described in the embodiment shown in FIG. 3A, and the dock module 138 is separated from the wearable device and substantially includes the neuromodulation controller 110 previously described in the embodiment shown in FIG. 3A. The plurality of electrodes 102 of the electrode module 101 may include a plurality of groups of electrodes (e.g., the groups 102A, 102B, 102C, 102D, and 102E). Each of these groups of electrodes 102 may have 16 electrode units. In some embodiments, a group of auxiliary electrodes (e.g., the group 102F) for EMG, REM etc., can be included, while such group of auxiliary electrodes may have 8 electrode units. In some embodiments, a reference electrode 102G can be included. These electrodes 102 are configured to obtain pathological activities of a patient with sleeping or movement disorder.

Still referring to FIG. 3D, in some embodiments, the plurality of electrodes 102 are coupled with the neuromodulation module 108. The neuromodulation module 108 is configured to record pathological activities and execute a stimulation through the plurality of electrodes simultaneously. In some embodiments, the neuromodulation module 108 includes a plurality of stimulation submodules 132A and a plurality of recording submodules 132B. These stimulation submodules 132A and these recording submodules 132B are configured to control the plurality of electrodes 102, including the functions of stimulation and recording. In some embodiments, the neuromodulation module 108 further includes a central control submodule (134A and 134B) configured to control the plurality of stimulation submodules 132A and the plurality of recording submodules 132B.

Since the electrode module 101 is used to provide stimulations to the brain of patient, while the electrode module 101 is also used to receive the brainwave of patient for recording, the electronic signals on the path of stimulation and the recording may interfere with each other if all the corresponding components are connected with an electrical wiring manner. In case interference exists, the brainwave obtained by the recording component can be affected, and so that the STW oscillation episodes may not be correctly identified. Therefore, in some embodiments, each of the stimulation submodules 132A is electrically isolated from each of the recording submodules 132B, and each of the stimulation submodules 132A is communicated with each of the recording submodules 132B by a non-electrical manner. For instance, within each group of the combination of the stimulation submodule 132A and the recording submodule 132B, the stimulation submodule 132A can communicate with the recording submodule 132B by using an optical mechanism 130. For example, the stimulation submodule 132A can optically couple with the recording submodule 132B by optical waveguides or photo-coupled ICs. In other examples, the stimulation submodule 132A can communicate with the recording submodule 132B by magnetic coupling technique.

In some embodiments, the central control submodule may have a stimulation central 134A utilized as a central unit for stimulation, whereas a recording central 134B of the central control submodule is utilized as a central unit for recording. In other words, the stimulation submodules 132A and the recording submodules 132B are peripheral units in the functions of stimulation and recording, these peripheral units are used for control specific group of electrodes (e.g., there are five stimulation submodules 132A and five recording submodules 132B for controlling the groups 102A, 102B, 102C, 102D, and 102E of the electrodes), while these peripheral units are all controlled by a single central unit (i.e., the central control submodule) Like the concern of the interference of the electrical signals, in some embodiments, the combination of the stimulation submodule 132A and the recording submodule 132B, the stimulation central 134A can communicate with the recording central 134B by using the optical mechanism 130. In some embodiments, the stimulation central 134A and the recording central 134B of the central control submodule may be wirelessly connected to the dock module 138, the dock module 138 is configured to optimize a parameter of the stimulation delivered by the plurality of electrodes. In some embodiments, the dock module 138 still may include several physical ports such as power jack or some USB ports for wiring connection.

Referring back to FIG. 3A and FIG. 3D, the MCU 114 in FIG. 3A performs the processing task in the central control submodule (134A and 134B), the stimulation submodules 132A, and the recording submodules 132B in FIG. 3D. In order to enhance parallel computation power, in some embodiments, the stimulation central 134A of the central control submodule may include or connect to a first AI core and/or the recording central 134B may include or connect to a second AI core. The first AI core and the second AI core can be used to enhance the computation power of the stimulation central 134A and the recording central 134B to improve the control over the peripheral submodule such as the stimulation submodule 132A and the recording submodule 132B.

Referring to FIG. 3A and FIG. 3D, the first connection submodule 136A and the second connection submodule 136B in FIG. 3D can be equivalent or identical to the first RF unit 116 in FIG. 3A as previously described. The first connection submodule 136A and the second connection submodule 136B transmit the data from stimulation central 134A and the data from recording central 134B, respectively, to the dock module 138. Likewise, the first connection submodule 136A and a second connection submodule 136B may also receive the signal or data from the dock module 138 and transmit to the stimulation central 134A and the recording central 134B, respectively. In some embodiments, the first connection submodule 136A and the second connection submodule 136B are communication modules for wireless connections, whereas in other embodiments, the first connection submodule 136A and the second connection submodule 136B may include some physical ports (e.g., USB ports) for wiring connection with the dock module 138.

Referring to FIG. 3E, in some embodiments, within the stimulation submodule 132A, the stimulation submodule 132A can include a first processing unit 144A, a biochip unit 142A, a power unit 140A, an impedance test unit 146A, and a true current unit 148. In some embodiments, the first processing unit 144A and the biochip unit 142A are substantially functioned as the MCU 114 and the biochip 112 previously disclosed in the embodiment shown in FIG. 3A. The power unit 140A is used to provide the system power. The impedance test unit 146A is used to provide impedance test. The true current unit 148 is used to monitor the true stimulation current delivered to an electrode. The recording submodule 132B is connected with the stimulation submodule 132A by the optical mechanism 130. The recording submodule 132B can include a second processing unit 144B, a biochip unit 142B, a power unit 140B, and an impedance test unit 146B. Other than the true current unit 148, the physical components in the recording module 132B is similar with those in the stimulation submodule 132A.

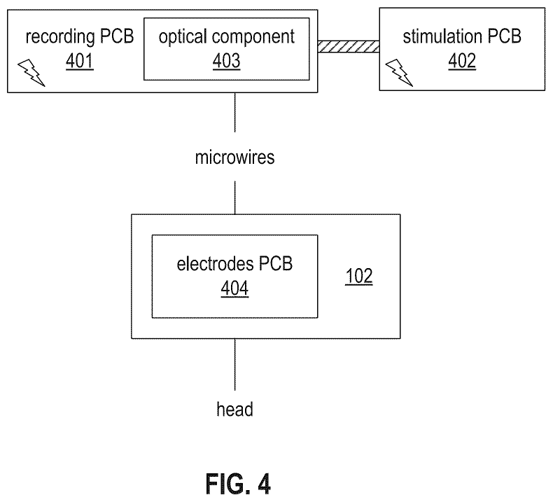

In some embodiments, referring to FIG. 4, the hardware layout design in the present disclosure can include a recording PCB 401 with an optical component 403 such as optical waveguides or photo-coupled integrated circuits, a stimulation PCB 402, and an electrode PCB 404. In some embodiments, the stimulation PCB 402 is electrically isolated from the recording PCB 401. Data or signal communication between the stimulation PCB 402 and the recording PCB 401 can be converted to and from optical form so as to reduce interference between the recording signal and the stimulation signal which is being processed in electrical form. The users can use the recording function alone by assuming the recording PCB 401 and the electrode PCB 404, or can use both the recording and the stimulation functions by assembling the recording PCB 401, the stimulation PCB 402 and the electrode PCB 404 together, if the recording and the stimulation functions are both required. The hardware utilizes a modular design, with each module supporting recording and stimulation through N (a particular natural number) electrodes 102. In some embodiments, the recording PCB 401 and the stimulation PCB 402 are separately-coupled with two different batteries for providing the power for recording and stimulating independently.

The foregoing outlines structures of several embodiments so that those skilled in the art may better understand the aspects of the present disclosure. Those skilled in the art should appreciate that they may readily use the present disclosure as a basis for designing or modifying other operations and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A method for treating sleeping or movement disorder, the method comprising:
recording a brainwave of a patient with sleeping or movement disorder;
identifying an onset of a sawtooth wave (STW) oscillation episode from the brainwave, wherein the STW oscillation episode is a STW signal having an oscillation frequency in a range of from about 2 Hz to about 4 Hz;
delivering a first stimulation to the patient when the onset of the STW oscillation episode is identified from the brainwave; and
adapting the first stimulation according to a measurable feature of the STW oscillation episode.

2. The method of claim 1, wherein the sleeping or movement disorder comprises rapid eye movement (REM) sleep behavior disorder (RBD), alpha-synucleinopathy (ASP), or Parkinson's disease (PD).

3. The method of claim 1, further comprising:
identifying whether the onset of the STW oscillation episode occurs in a REM stage or an awake stage of the patient; and
delivering the first stimulation to the patient only when (1) the onset of the STW oscillation episode occurs during awake stage of the patient, or (2) the onset of the STW oscillation episode occurs during the REM stage of the patient and in excess of a predetermined occurrence frequency.

4. The method of claim 1, wherein the first stimulation is applied during a course of the STW oscillation episode, and the first stimulation is terminated when the STW signal is decayed to a lower threshold, terminated, or after the STW signal being terminated for 1.0 to 2.0 seconds.

5. The method of claim 1, wherein the measurable feature of the STW oscillation episode comprises an oscillation intensity, the oscillation frequency, an energy derived from the STW oscillation episode, an oscillation duration, a frequency of occurrence of the STW oscillation episode, or the combinations thereof.

6. The method of claim 1, wherein the onset of the STW oscillation episode is identified from the brainwave of the patient by monitoring electroencephalography (EEG), electromyography (EMG), and electro-oculography (EOG) recordings simultaneously.

7. The method of claim 1, wherein the first stimulation comprises multiple intermittent sub-stimulations during the STW oscillation episode.

8. The method of claim 1, further comprising:
recording the measurable feature of the STW oscillation episode before or during delivering the first stimulation, the measurable feature comprises at least one of an oscillation intensity, the oscillation frequency, an oscillation duration of the STW oscillation episode, or a frequency of occurrence of the STW oscillation episode within a predetermined timeframe.

9. The method of claim 8, further comprising:
delivering a second stimulation subsequent to terminating the first stimulation when an onset of another STW oscillation episode is identified in the brainwave, wherein the second stimulation is applied with the stimulation form determined by at least one of the oscillation duration of the STW oscillation episode or the frequency of occurrence of the STW oscillation episode within the predetermined timeframe.

10. The method of claim 1, further comprising:
pausing the first stimulation during the course of the oscillation episode before the first stimulation is terminated.

11. A system for treating sleeping or movement disorder, comprising:
an electrode module (101), comprising:
a plurality of electrodes, configured to obtain pathological activities of a patient with sleeping or movement disorder; and
a neuromodulation module coupled with the plurality of electrodes, configured to record pathological activities and execute a stimulation through the plurality of electrodes simultaneously, and
a dock module wirelessly connected to the electrode module, configured to optimize a parameter of the stimulation,
wherein the pathological activities comprises a sawtooth wave (STW) demonstrating at least one oscillation episode each having an oscillation frequency in a range of from about 2 Hz to about 4 Hz.

12. The system of claim 11, wherein the electrode module is a wearable device, and the dock module is separated from the wearable device.

13. The system of claim 11, wherein the neuromodulation module comprises:
a plurality of stimulation submodules and a plurality of recording submodules configured to control the plurality of electrodes;
a central control submodule configured to control the plurality of stimulation submodules and the plurality of recording submodules.

14. The system of claim 13, wherein each of the stimulation submodules is electrically isolated from each of the recording submodules.

15. The system of claim 13, wherein each of the stimulation submodules is communicated with each of the recording submodules by a non-electrical manner.

16. The system of claim 11, wherein the plurality of electrodes each further comprises an amplifier module configured to amplify a brainwave of the patient.

17. The system of claim 13, wherein the stimulation submodules comprises a first processing unit and the recording submodules comprises a second processing unit.

18. The system of claim 17, wherein a stimulation central of the central control submodule comprises a first AI core or a recording central of the central control submodule comprises a second AI core.

19. The system of claim 11, wherein the dock module comprises a third AI core.

20. The system of claim 19, wherein the third AI core is configured to implement a computationally-intensive Kalman filter for maintaining detection among different durations or patients.
